(19) United States
(12) Patent Application Publication
(10) Pub. No.: US 2011/0118734 A1
(43) Pub. Date: May 19, 2011

(54) CAPSULARHEXIS DEVICE USING PULSED ELECTRIC FIELDS

(75) Inventors: Jack R. Auld, Laguna Niguel, CA (US); Tammo Heeren, Aliso Viejo, CA (US); John C. Huculak, Mission Viejo, CA (US); Steven W. Kovalcheck, Aliso Viejo, CA (US)

(73) Assignee: ALCON RESEARCH, LTD., Fort Worth, TX (US)

(21) Appl. No.: 12/618,805

(22) Filed: Nov. 16, 2009

(51) Int. Cl. A61B 18/18

(52) U.S. Cl. 606/45

(57) ABSTRACT

A capsulorhexis apparatus is disclosed. An exemplary apparatus includes a cutting electrode device that in turn comprises a handle, a flexible ring having a single ring-shaped wire electrode embedded therein, and a shaft connecting the flexible ring to the handle, wherein the flexible ring is configured for insertion into an eye through an incision. The apparatus further includes a grounding electrode configured for placement in or on the eye, independently of the cutting electrode device, and a pulse generator electrically connected to the ring-shaped wire electrode and the grounding electrode and configured to supply pulsed power to the eye via the ring-shaped wire electrode and the grounding electrode

### TECHNICAL FIELD

The present invention relates generally to the field of cataract surgery and more particularly to methods and apparatus for performing a capsulorhexis.

### BACKGROUND

An accepted treatment for the treatment of cataracts is surgical removal of the lens and replacement of the lens function by an artificial intraocular lens (IOL). In the United States, the majority of cataractous lenses are removed by a surgical technique called phacoemulsification. Prior to removing the cataractous lens, an opening, or rhexis, must be made in the anterior capsule. During phacoemulsification, there is a great deal of tension on the cut edges of the anterior capsulorhexis while the lens nucleus is emulsified. Accordingly, a continuous cut or tear (rhexis), without “tags,” is a critical step in a safe and effective phacoemulsification procedure.

If the capsule is opened with numerous small capsular tears, the small tags that remain can lead to radial capsular tears which may extend into the posterior capsule. Such a radial tear constitutes a complication since it destabilizes the lens for further cataract removal and safe intraocular lens placement within the lens capsule later in the operation. Further, if the posterior capsule is punctured then the vitreous may gain access to the anterior chamber of the eye. If this happens, the vitreous must be removed by an additional procedure with special instruments. The loss of vitreous is also associated with an increased rate of subsequent retinal detachment and/or infection within the eye. Importantly, these complications are potentially blinding.

Conventional equipment used for phacoemulsification includes an ultrasonically driven handpiece with an attached cutting tip. In some of these handpieces, the operative part is a centrally located, hollow resonating bar or horn directly attached to a set of piezoelectric crystals. The crystals supply ultrasonic vibration for driving both the horn and the attached cutting tip during phacoemulsification.

Prior art devices and methods used for the capsulorhexis procedure require a great deal of skill on the part of the surgeon to produce a continuous curvilinear capsular opening. This is due to the extreme difficulty in controlling the path of the cutting tip of the device. For example, a typical procedure begins with a capsular incision made with a cystotome, e.g., a cutting tip as described above. This incision is then coaxed into a circular or oval shape by pushing the leading edge of the incision in the capsule, using the cystotome as a wedge rather than in a cutting fashion. Alternatively, the initial capsular incision may be torn into a circular shape by grasping the leading edge with fine caliber forceps and advancing the cut. Either of these approaches involves a very challenging maneuver and the tearing motion can sometimes lead to an undesirable tear of the capsule toward the back of the lens, even in the most experienced hands.

Moreover, even if a smooth capsular opening without tags is ultimately produced, the size and/or position of the capsular opening may present a problem. For instance, a capsular opening that is too small can impede the safe removal of the lens nucleus and cortex and prevent proper intraocular lens insertion into the lens capsule. The additional stresses necessary to accomplish the operation with a small or misplaced capsular opening put the eye at risk for zonular and capsular breakage. Either of these complications will likely increase the length and complexity of the operation and may result in vitreous loss.

A continuous, properly positioned, and circular opening is thus highly desirable because it results in: (1) a significant reduction in radial tears and tags within the anterior capsule, (2) capsule integrity necessary for proper centering of a lens prosthesis; (3) safe and effective hydrodissection; and (4) safe use of capsular procedures on patients having poorly visualized capsules and/or small pupil openings. In addition, the capsulorhexis should be properly dimensioned relative to the diameter of the IOL being implanted in order to reduce the chances of a secondary cataract, also called posterior capsule opacification (“PCO”) and for use with proposed accommodative IOLs designs. Therefore, there is a continuing need for improved devices for performing anterior chamber capsulorhexis.

### SUMMARY

As described more fully below, embodiments of the present invention include a capsulorhexis apparatus, including a cutting electrode device that in turn comprises a handle, a flexible ring having a single ring-shaped wire electrode embedded therein, and a shaft connecting the flexible ring to the handle, wherein the flexible ring is configured for insertion into an eye through an incision. Various embodiments of the invention further comprise a grounding electrode configured for placement in or on the eye, independently of the cutting electrode device, and a pulse generator electrically connected to the ring-shaped wire electrode and the grounding electrode and configured to supply pulsed power to the eye via the ring-shaped wire electrode and the grounding electrode. Some embodiments further comprise a tubular insertion cartridge configured to fit around the shaft and to substantially contain the flexible ring when the flexible ring is in a retracted position.

In some embodiments, the grounding electrode comprises a substantially smooth coin-shaped electrode, while in others, the grounding electrode comprises a substantially smooth paddle-shaped electrode. The ring-shaped wire element has a thin cross-section, e.g., less than about 0.25 mm, so that high-intensity electric fields are created in the eye at or near the ring-shaped wire element.

Methods for performing capsulorhexis are also described, including methods that comprise inserting a flexible ring having a single ring-shaped wire electrode embedded therein into the anterior chamber of an eye, positioning the flexible ring in contact with the anterior lens capsule of the eye, positioning a grounding electrode in or on the eye, and supplying pulsed power to the eye via the ring-shaped wire electrode and the grounded electrode.

In some embodiments, inserting the flexible ring into the anterior chamber of the eye comprises inserting the distal end of a tubular insertion cartridge into the anterior chamber, through an incision in the eye, and ejecting the flexible ring into the anterior chamber from a retracted position wherein the flexible ring is substantially contained within the tubular insertion cartridge. The incision may be less than about 2 millimeters, in some embodiments.

Of course, those skilled in the art will appreciate that the present invention is not limited to the above features, advantages, contexts or examples, and will recognize additional features and advantages upon reading the following detailed description and upon viewing the accompanying drawings.

### BRIEF DESCRIPTION OF THE DRAWINGS

FIG. 1 illustrates a capsulorhexis apparatus according to some embodiments of the invention, including a pulse generator, cutting electrode device, and grounding electrode device.

FIG. 2 illustrates details of an exemplary cutting electrode device.

FIG. 3 is a cross-section of the ring portion of the cutting electrode device of FIG. 2.

FIG. 4 illustrates details of an exemplary grounding electrode device.

FIG. 5 is a schematic diagram illustrating functional elements of an exemplary pulse generator.

FIG. 6 illustrates the use of a cutting electrode device and ground electrode in an eye.

FIG. 7 is a process flow diagram illustrating an exemplary method for using an autocapsulorhexis system.

### DETAILED DESCRIPTION

Various embodiments of the present invention provide apparatus and corresponding methods of use for performing capsulorhexis. In particular, the present invention relates to a surgical instrument, a flexible capsulorhexis electrode device, which may be positioned within the anterior chamber of an eye through a small incision to perform capsulorhexis, or capsulotomy. This procedure facilitates phacoemulsification of a cataractous lens and insertion of an artificial intraocular lens (IOL).

Various methods and devices for automating the capsularhexis process have been proposed. United States Patent Application Publication No. 2006/0100617, the entire contents of which are incorporated herein by reference, describes an “autocapsulorhexis” device comprising a circular, flexible ring made of an elastomer or an acrylic or thermoplastic material. Embedded within each of various embodiments of this flexible ring is either a resistance-heating element or a pair of bipolar electrodes, which are energized according to known techniques to produce localized heating on the anterior capsule, so as to define a weakened boundary for an easy detachment of the portion of the capsule within the circular ring. Various other devices have been proposed, many of which depend on resistive-heating cautery elements, such as U.S. Pat. No. 6,066,138, issued May 23, 2000; U.S. Pat. No. 4,481,948, issued Nov. 13, 1984; and WIPO Publication No. WO 2006/109290 A2, published Oct. 19, 2006. The entire contents of each of the previous references are incorporated by reference herein, for the purpose of providing background and context for the present invention.

Although those skilled in the art will appreciate the broader applicability of several of the inventive techniques and apparatus disclosed herein, the present invention is generally directed to methods and apparatus for performing capsulorhexis using high-frequency electrical currents applied to the anterior lens capsule through a unipolar electrode. A path for return currents is provided through a separately applied grounding electrode, as described in further detail below.

The device uses pulsed electric fields to perform the cutting action—the pulsed electric field is generated using a ring electrode, placed against the anterior capsule of the eye, and a grounding electrode located at a different position inside or outside the eye. In many embodiments, the ring electrode comprises a thin, electrically conducting wire. A very thin wire will increase cutting efficiency and reduce far-field effects. A very small cross-section (e.g., less than about 0.25 millimeters in diameter) will yield high-intensity electric fields close to the wire; these electric fields will reduce in intensity further away from the wire. Because the ground electrode has a much larger cross-section than the cutting electrode, the electric fields remain attenuated at the grounding electrode. Thus, a high proportion of the available cutting energy is deposited into a thin region immediately around the cutting electrode's wire.

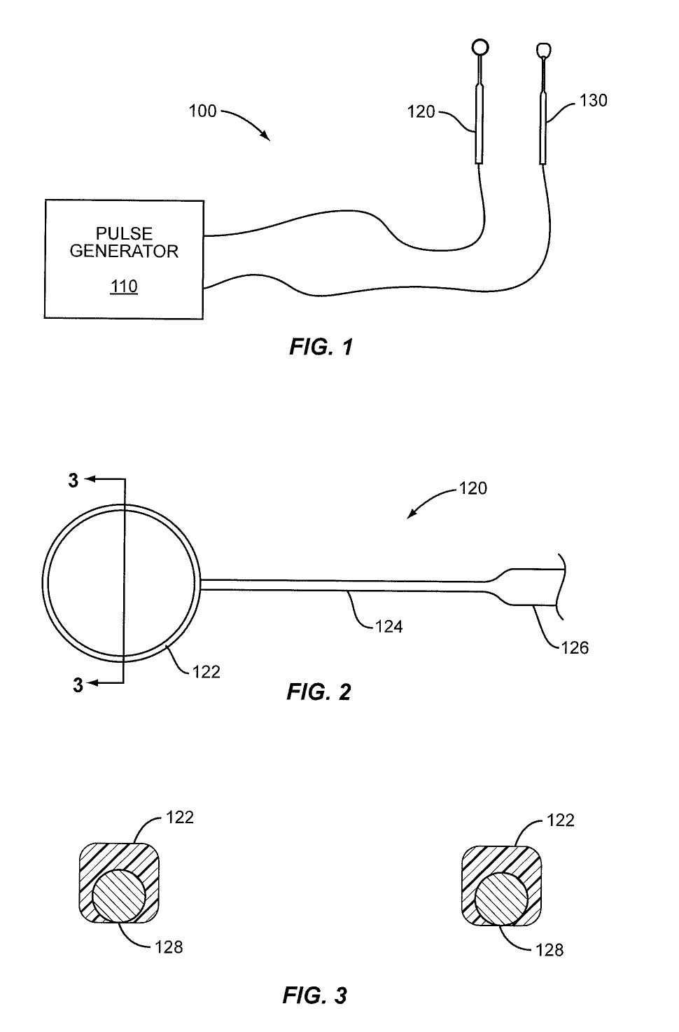

FIG. 1 illustrates the components of an exemplary capsulorhexis apparatus according to some embodiments of the invention. The pictured system includes a pulse generator 110, which produces high-frequency pulses for application to the eye through cutting electrode 120 and grounding electrode 130.

FIGS. 2 and 3 illustrate details of an exemplary cutting electrode device 120. Cutting electrode device 120 includes a flexible ring 122, which a single, ring-shaped, wire electrode 128 embedded therein. A flexible shaft 124 connects the flexible ring 122 to a handle 126. An electrical lead (not shown) runs within shaft 124 and handle 126 to connect electrode 128 to the pulse generator 110.

Various structures for the cutting electrode device are possible. Several approaches to fabricating such a device are described in United States Patent Application Publication No. 2006/0100617 (incorporated by reference above). Another approach is to form the electrode by depositing electrically conductive ink onto an elastomeric ring to form a conductive trace, e.g., by insert molding the elastomeric substrate and then screen-printing conductive traces to the desired dimensions. Alternatively, an adhesive trace can be applied to a pre-fabricated elastomeric ring, or a conductive trace can be combined with an elastomeric ring through an insert molding process.

In any case, the flexible ring portion of the apparatus is dimensioned according to the desired size of the capsulotomy, e.g., with a diameter of approximately 5 millimeters. Those skilled in the art will appreciate that a circular opening is preferred, as illustrated in FIG. 2, to avoid tearing when the portion of the lens capsule within the opening is removed. Of course, those skilled in the art will appreciate that some variation from a circular shape may be acceptable in some applications. Thus, the term “ring” as used herein will be understood to include generally circular, oval, or elliptical structures.

The ring-shaped wire electrode 128 defines the boundaries of the portion of the lens capsule that is subjected to the high-frequency electric field when the electrode is energized. The basic principles of such electro-surgery, which may involve, for example, frequencies of greater than 100 kHz, are well known to those skilled to the art. Accordingly, the details of such procedures, which are not necessary to a complete understanding of the present invention, are not provided herein.

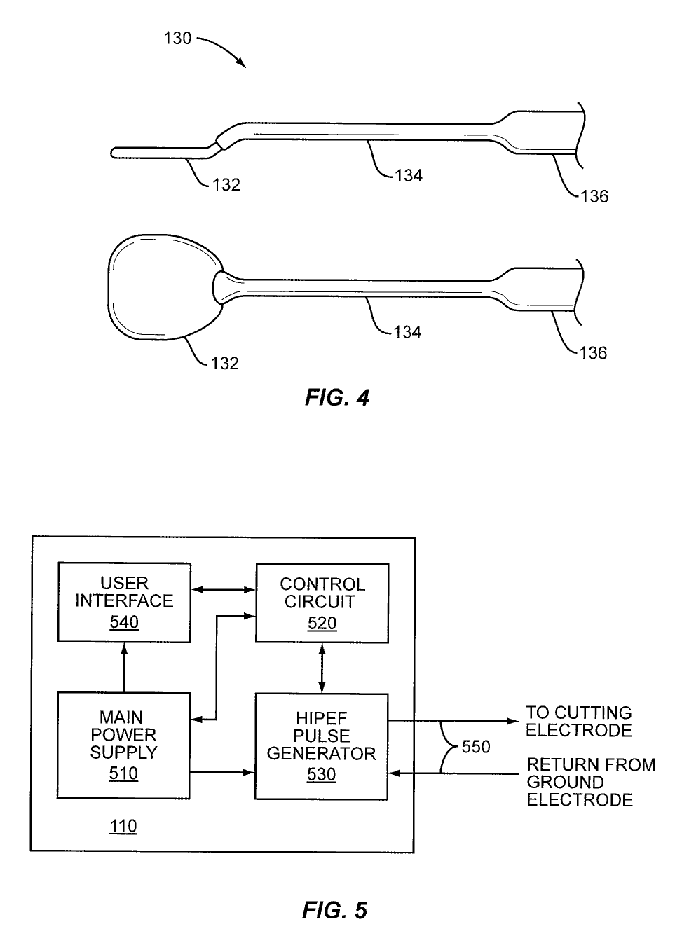

As noted above, return currents from the high-intensity pulsed electric field applied to the cutting electrode device 120 flow through a grounding electrode independently inserted into or placed on the eye. Although the grounding electrode may take any of a number of shapes and sizes suitable for placement in or on the eye, FIG. 4 illustrates one exemplary configuration of a grounding electrode device 130. In the pictured embodiment, grounding electrode 130 includes a paddle-shaped conductor 132, a shaft 134, and a handle 136. The paddle-shaped conductor 132 is shaped and dimensioned to be placed in or on the eye, and comprises a conductive electrode with a cross-section and surface area substantially greater than those of the cutting electrode device; this, coupled with a substantially smooth surface, keeps the electric fields at or near the electrode at a relatively low intensity, minimizing far-field effects from the high-intensity pulsed electric fields applied to the cutting electrode. The shaft 134 may be flexible, in some embodiments, to facilitate insertion into the eye. The shaft 134 and 136 contain an electrically conductive lead (not pictured) to connect the grounding electrode 132 to the pulse generator 110.

Those skilled in the art will appreciate that the shape of the grounding electrode may vary. In addition to the paddle shape shown in FIG. 4, other possible shapes include a coin, or disc, shape, or doughnut (toroid) shape. In general, the surface of the grounding electrode may be substantially smooth, i.e., sharp angles and small dimensions should be avoided, to minimize high-intensity fields at or near the grounding electrode.

FIG. 5 illustrates functional elements of a pulse generator 110 according to some embodiments of the present invention. Pulse generator 110 includes a main power supply 510, which may be operated from an external alternating current source (e.g., 120 volts at 60 Hz) or direct current source. HIPEF pulse generator 530 generates the high-intensity pulses, from the main power supply 510, under the control of control circuit 520. The high-intensity pulses are supplied to the cutting electrode device 120 and grounding electrode device 130 through leads 550. User interface 540 provides the operator with appropriate mechanisms for operating the pulse generator 110 (e.g., switches, touch-screen inputs, or the like), as well as appropriate feedback (e.g., device status, etc.). Further details of a high-intensity pulsed electric field generator apparatus that can readily be adapted for the present application are provided in U.S. Patent Application Publication 2007/0156129 A1, published 5 Jul. 2007, the entire contents of which are incorporated herein by reference.

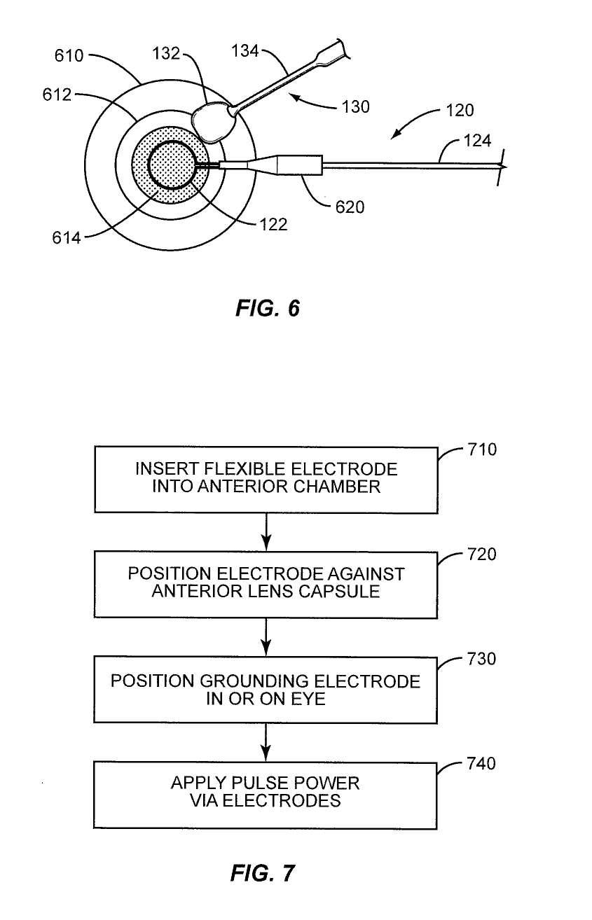

FIG. 6 illustrates the use of a cutting electrode device 120 and ground electrode 130 in an eye 610, while FIG. 7 is a process flow diagram illustrating an exemplary method of use. As shown at block 710 of FIG. 710, the flexible electrode 122 is inserted into the anterior chamber 612 of the eye 610 via a small incision (e.g., 2 millimeters or less) in the eye 610. In some cases, the flexible electrode 122 may be inserted by way of a tubular insertion cartridge 620. In these embodiments the flexible electrode 122 may be pre-inserted into the cartridge 620 before use; i.e., the flexible ring 122 begins in a retracted position such that the flexible ring 122 is contained substantially within the insertion cartridge 620. The distal end of the tubular insertion cartridge 620 is inserted into the eye 610 through the incision, and the flexible ring 122 is ejected into the anterior chamber by pushing the shaft 124 through the cartridge 620.

In any case, the ring-shaped electrode 122 is then placed against the surface of the lens capsule 614, as shown at block 720 of FIG. 7. The ground electrode portion 132 of the grounding electrode device 130 is positioned in or on the eye to provide a return path for the high-intensity pulsed electric field currents, as shown at block 730, and pulsed power is applied to the eye via the cutting electrode device 120 and the grounding electrode device 130. After energizing the apparatus, the capsule area defined by the ring-shaped electrode 122 is weakened, and subject to easy removal using conventional forceps.

In some embodiments the cutting electrode device 120, or the grounding electrode device 130, or both, are detachable from one or more cables running to and from the pulse generator 110. Thus, the two devices may be removed from the tool, which allows the devices to be sterilized and reused multiple times. In other embodiments, one or both devices may be disposable, i.e., intended to be used only once or a few times.

The preceding descriptions of various embodiments of capsulorhexis apparatus and methods for utilizing these apparatus were given for purposes of illustration and example. Those skilled in the art will appreciate, of course, that the present invention may be carried out in other ways than those specifically set forth herein without departing from essential characteristics of the invention. The present embodiments are thus to be considered in all respects as illustrative and not restrictive, and all changes coming within the meaning and equivalency range of the appended claims are intended to be embraced therein.

What is claimed is:

1. A capsulorhexis apparatus, comprising:
a cutting electrode device comprising a handle, a flexible ring having a single ring-shaped wire electrode embedded therein, and a shaft connecting the flexible ring to the handle, wherein the flexible ring is configured for insertion into an eye through an incision;
a grounding electrode configured for placement in or on the eye, independently of the cutting electrode device; and
a pulse generator electrically connected to the ring-shaped wire electrode and the grounding electrode and configured to supply pulsed power to the eye via the ring-shaped wire electrode and the grounding electrode.

2. The capsulorhexis apparatus of claim 1, further comprising a tubular insertion cartridge configured to fit around the shaft and to substantially contain the flexible ring when the flexible ring is in a retracted position.

3. The capsulorhexis apparatus of claim 1, wherein the grounding electrode comprises a substantially smooth coin-shaped electrode.

4. The capsulorhexis apparatus of claim 1, wherein the grounding electrode comprises a substantially smooth paddle-shaped electrode.

5. The capsulorhexis apparatus of claim 1, wherein the ring-shaped wire electrode has a maximum cross-sectional dimension of about 0.25 mm.

6. A method of performing capsulorhexis, the method comprising:
inserting a flexible ring having a single ring-shaped wire electrode embedded therein into the anterior chamber of an eye;
positioning the flexible ring in contact with the anterior lens capsule of the eye;
positioning a grounding electrode in or on the eye; and
supplying pulsed power to the eye via the ring-shaped wire electrode and the grounded electrode.

7. The method of claim 6, wherein inserting the flexible ring into the anterior chamber of the eye comprises:
inserting the distal end of a tubular insertion cartridge into the anterior chamber, through an incision in the eye; and
ejecting the flexible ring into the anterior chamber from a retracted position wherein the flexible ring is substantially contained within the tubular insertion cartridge.

8. The method of claim 7, wherein the incision is less than about 2 millimeters.
